(19) United States
(12) Patent Application Publication
(10) Pub. No.: US 2025/0052750 A1
(43) Pub. Date: Feb. 13, 2025

(54) METHOD FOR DETERMINING BASOPHIL ACTIVATION IN A SAMPLE

(71) Applicant: Deutsches Rheuma-Forschungszentrum Berlin, Berlin (DE)

(72) Inventors: Ria Baumgrass, Berlin (DE); Johannes Groffmann, Berlin (DE)

(73) Assignee: Deutsches Rheuma-Forschungszentrum Berlin, Berlin (DE)

(21) Appl. No.: 18/722,884

(22) PCT Filed: Dec. 23, 2022
(86) PCT No.: PCT/EP2022/087750

§ 371 Date: Jun. 21, 2024

(30) Foreign Application Priority Data
Dec. 23, 2021 (EP) 21217298.5

(51) Int. Cl. G01N 33/569; G01N 33/564; G16B 50/20

(52) U.S. Cl. CPC G01N 33/56972; G01N 33/564; G16B 50/20; G01N 2333/70535; G01N 2333/70596; G01N 2800/52

(57) ABSTRACT

An in vitro method for determining basophil activation in a sample is provided. Other aspects relate to determining an efficacy of an allergen-specific desensitization treatment of a subject. The method includes providing a sample from the subject, treating the sample with an allergen and determining a level of at least one basophil inhibitory marker and at least one basophil activation marker in the sample, the basophil inhibitory marker includes CD32. Corresponding systems and machine learning methods are also described. The use of an affinity reagent that specifically binds CD32 in an in vitro method for determining basophil activation is also provided, for example in the context of determining an efficacy of an allergen-specific desensitization treatment of a subject. A kit and corresponding uses of affinity reagents is also detailed.

The present invention is in the field of allergy diagnostics and in vitro methods for determining basophil activation in a sample, and for determining and/or prognosing the efficacy of an allergen-specific desensitization treatment.

The invention relates to an in vitro method for determining basophil activation in a sample obtained from a subject, comprising treating said sample with an allergen and determining a level of at least one basophil inhibitory marker and at least one basophil activation marker in the sample, wherein the basophil inhibitory marker comprises CD32.

The invention relates further to an in vitro method for determining and/or prognosing an efficacy of an allergen-specific desensitization treatment of a subject, comprising providing a sample from said subject, treating said sample with an allergen and determining a level of at least one basophil inhibitory marker and at least one basophil activation marker in the sample.

The invention relates further to a machine learning-supported evaluation of basophil activation or evaluating the efficacy of an allergen-specific desensitization treatment of a subject.

In another aspect, the invention relates to a system for determining basophil activation in a sample, or determining and/or prognosing the efficacy of an allergen-specific desensitization treatment of a subject, comprising a) a data store comprising multiple feature sets; and b) a software configured for generating the feature sets and comparing the feature sets.

The invention relates to the use of an affinity reagent that specifically binds CD32 in an in vitro method for determining basophil activation in a sample, or determining and/or prognosing an efficacy of an allergen-specific desensitization treatment of a subject, preferably in combination with the use of an affinity reagent that specifically binds CD63.

The invention further relates to a kit for determining basophil activation in a sample, or determining and/or prognosing the efficacy of an allergen-specific desensitization treatment of a subject, comprising an allergen against which the subject exhibits a hypersensitivity reaction; and at least one label that specifically binds a basophil inhibitory marker and at least one label that specifically binds a basophil activation marker.

### BACKGROUND OF THE INVENTION

Allergen-specific immunotherapy (desensitization) is a treatment used by allergists and immunologists for common allergic conditions, such as allergic rhinitis/conjunctivitis, allergic asthma and stinging insect hypersensitivity, amongst other conditions. This form of therapy typically involves the subcutaneous administration of gradually increasing quantities of the relevant allergens until a dose is reached that is effective in inducing immunologic tolerance to the allergens. The primary objectives of allergen-specific immunotherapy are to decrease the symptoms triggered by allergens and to prevent recurrence of the disease in the long-term.

Currently, immunotherapies are the most effective therapies for many allergies, to minimize excessive immune reactions to antigens that are actually harmless (Allergy, Incorvaia 2020 et a., personalized medicine for allergy treatment: allergen immunotherapy still a unique and unmatched model).

However, the therapeutic effect is still not ideal. Local and systemic reactions may occur. Local reactions, such as redness or itching at the injection site, can generally be managed with local treatment (e.g., cool compresses or topical corticosteroids) or oral antihistamines. Systemic reactions to allergen immunotherapy occur in approximately 1-4% of patients and can be mild to severe. The most severe reaction is anaphylaxis. Fatal anaphylactic reactions are rare, occurring in an estimated 1 in every 8 million doses of immunotherapy administered. Furthermore, the efficacy of the treatment, i.e. the effectiveness in reducing the allergy in the subject, varies depending on the subject and in some cases is lower than desired.

Hence, the efficacy of any given allergen-specific immunotherapy needs to be determined from time to time before therapy initiation, during therapy and even after termination of the therapy, in order to determine whether the therapy is advisable and effective. Unfortunately, there are currently no in vitro tests that can reliably diagnose the success of such therapy.

Established skin prick tests (SPT) used for this purpose and the immunoglobulin IgE antibody assays cannot replace a potentially life-threatening provocation test at the end of treatment, which usually lasts three years. The disadvantages of skin prick tests are, for example, that systemic and topical antihistamines may suppress the weal and flare reaction; the test is less reliable with food allergens (which are less well standardized) than with inhaled allergens, itching causes a slight discomfort, and interpretation is difficult in patients with eczema or dermographism.

The basophil activation test (BAT), for example, using surface markers CCR3 with CD63, or CD63 with CD203c, which have been used in research so far, are also not ideal for clinical routine in its current form and does not distinguish between stable and transient responders.

US2020011865A1 discloses a method for determining basophil activation by measuring cell surface expression of CD123 and FcεRI, or CD123 and CD203c, or CD123 and HLA-DR, after contacting the sample with an allergen extract.

Santos et al. reveals that basophil CD123 expression decreases after stimulation with allergen extracts. The decrease in fluorescence intensity of CD123 correlates with an upregulation of basophil activation markers CD63 and CD203c (Santos et al: “The expression of CD1 23 can decrease with basophil activation: implications for the gating strategy activation test”, Clinical and Translational Allergy, vol. 6, no. 1, 1 Apr. 2016, p. 1-11). However, CD123 is not considered a basophil inhibitory marker, as CD123 is not an inhibitor of basophil activity itself, rather it merely decreases in amount upon stimulation.

Oliver et al. describes that an inhibitory crosstalk is established between FcεRI and a co-expressed receptor FcγRII (CD32), by FcγRII suppressing Syk activation mediated by FcεRI (Oliver J M et al: “Immunologically mediated signaling in basophils and mast cells: finding therapeutic targets for allergic diseases in the human fcepsilonr1 signaling pathway”, Immunopharmacology, Elsevier Science Publishers BV, vol. 48, January 2000, p. 269-281).

Cady et al. discloses that FcγRIIA and FcγRIIB function cooperatively in activating the inhibitory pathway in allergen immunotherapy. FcγIIB expression is lower in patients undergoing allergen immunotherapy compared with nonallergic controllers (Cady C T et al: “IgG antibodies produced during subcutaneous allergen immunotherapy mediate inhibition of basophil activation via a mechanism involving both FcγRIIA and FcγRIIB”, Immunology letters, Elsevier B V, NL, vol. 130, no. 1-2, 4 May 2010, p. 57-65).

Tordesillas et al describes mass cytometric profiling of basophil and total peripheral blood compartment response to peanut using 30 antibodies, including anti-CD32, CD63, CD66b, HLA-DR, and FcεRI antibodies. (Tordesillas Leticia et al: “Mass cytometry profiling the response of basophils and the complete peripheral blood compartment to peanut”, Journal of Allergy and Clinical Immunology, 1 Dec. 2016, p.1-8). However, a method for determining basophil activation in a subject using CD32 is neither disclosed nor suggested.

Despite developments in the art with respect to in vitro diagnostics for therapy success, further improvements are required in providing sensitive, safe, reliable and reproducible in vitro tests for determining basophil activation in a sample, for example in the context of determining the efficacy of allergen specific immunotherapy.

Conventional data analysis of flow cytometry data, especially relating to the basophil activation assay, is based upon the principle of manual gating, which is repetitive and labor-intensive due to repeated sub-gating, lacks transparency and susceptible to bias. No consensus has been found as to which of many BAT protocols should be applied to obtain comparable results through their harmonization.

Patil et al 2018 proposes a data-driven programmatic approach to the analysis of basophil activation tests. An algorithm for autogating has been developed based on a subset of the BAT experiments from clinical trials. It was validated with the standard manual analysis of the same data, and then applied with an algorithm to a larger dataset. However, in this article, using an automated gating strategy, basophils were defined as bivariate normally distributed SSC^{low}/CCR3^{high} cells. The autogating strategy is therefore limited to a combination of two fluorescent markers (Cytometry Part B (clinical cytometry) 94B:667-673 (2018), data-driven programmatic approach to analysis of basophil activation tests).

Similar to Patil et al., Behrends et al. used a conventional 2 parameter gating strategy for identification of basophils (CD203C^{high}/FcεRI^{high}) based on the sums of the expression values for both parameters (Behrends J, Schwager C, Hein M, Scholzen T, Kull S, Jappe U. (2021) Innovative robust basophil activation test using a novel gating strategy reliably diagnosing allergy with full automation. Allergy 76, 3776-3788). Furthermore, in Patil et al and Behrends et al, only a basophil population is selected for analysis and other cell population are disregarded.

An earlier publication from the inventor of the present invention, WO 2020/201443, relates to a method for classifying selected marker signals from cytometric measurements. The method is suitable for a number of combinations of two or more fluorescent markers whose complexities for data analysis are dramatically raised. Cell subpopulations are not outgated but rather grouped and defined as bin areas using two or more cell marker intensities. The machine learning model trained with this method is able to autogate and/or visualize cell populations exhibiting a combination of signal intensities of two or more cell markers.

Despite some advances in the prior art, to the knowledge of the inventor, there has been no suggestion in the art until now to develop a bin-based automatic workflow for flow cytometry data analysis for the basophil activation assay, in which an analysis of the basophil population is carried out for a combination of two or more cell markers.

### SUMMARY OF THE INVENTION

In light of the prior art the technical problem underlying the present invention is to provide improved or alternative means for determining and/or prognosing the efficacy of an allergen-specific desensitization treatment.

This problem is solved by the features of the independent claims. Preferred embodiments of the present invention are provided by dependent claims.

The invention therefore relates to an in vitro method for determining basophil activation in a sample obtained from a subject, comprising treating said sample with an allergen and determining a level of at least one basophil inhibitory marker and at least one basophil activation marker in the sample, wherein the basophil inhibitory marker comprises CD32.

The invention relates further to an in vitro method for determining and/or prognosing an efficacy of an allergen-specific desensitization treatment of a subject, comprising providing a sample from said subject, treating said sample with an allergen and determining a level of at least one basophil inhibitory marker and at least one basophil activation marker in the sample.

The invention relates further to a machine learning-supported evaluation of the efficacy of an allergen-specific desensitization treatment of a subject as well as a bin-based algorithm.

The invention relates further to a system for determining and/or prognosing the efficacy of an allergen-specific desensitization treatment of a subject comprising a) a data store comprising multiple feature sets; and b) a software configured for generating the feature sets, and comparing the feature sets, wherein the software is configured to apply a model as described in more detail below.

The invention relates further to the use of an affinity reagent that specifically binds CD32 in an in vitro method for determining and/or prognosing an efficacy of an allergen-specific desensitization treatment of a subject, preferably in combination with the use of an affinity reagent that specifically binds CD63.

The invention relates further to a kit for determining and/or prognosing the efficacy of an allergen-specific desensitization treatment of a subject in a method as described herein, comprising an allergen against which the subject exhibits a hypersensitivity reaction; and at least one label that specifically binds a basophil inhibitory marker and at least one label that specifically binds a basophil activation marker.

The various aspects of the invention are unified by, benefit from, are based on and/or are linked by the surprising finding, common to all aspects of the invention, that an in vitro method for determining and/or prognosing an efficacy of an allergen-specific desensitization treatment can be improved by assessing a level of at least one basophil inhibitory marker and at least one basophil activation marker in the sample. Until the present time, to the knowledge of the inventors, this combination of marker classes has not been assessed in combination in the context of determining the efficacy of such a treatment.

Although the conventional basophil activation test (BAT) employs for example CCR3 and CD63 (as a basophil activation marker), it does not allow classification nor of responders and non-responders to immunotherapy.

Of further note, is that conventional BATs do not interrogate basophil inhibitory markers and as such provide an incomplete assessment of the basophil reactivity to allergen stimulus.

Surprisingly, a basophil inhibitory marker can also be applied to the BAT together with a basophil activation marker. The expression of such a basophil inhibitory marker is usually downregulated upon the activation of basophils after being incubated with the allergen, to which the subject is allergic.

On the basis of this surprising finding, the combination of at least a basophil activation marker and at least a basophil inhibitory marker renders the BAT more reliable and accurate in assessing the immune response in the subject. The combination of two kinds of basophil markers characterizes the BAT in different aspects. The disadvantage of only using basophil activation marker and/or basophil identification marker can be compensated by applying a basophil inhibitory marker. The determination of the efficacy of allergen-specific desensitization treatment is thus more reliable by means of applying a combination of using a basophil inhibitory marker and a basophil activation marker.

In one embodiment, the method as described herein comprises:
incubating a blood sample from said subject with an allergen, against which the subject exhibits or is suspected to exhibit a hypersensitivity reaction, wherein the sample comprises basophils;
treating the sample with at least one label that specifically binds a basophil inhibitory marker and at least one label that specifically binds a basophil activation marker; and
determining the signal intensities of the labels in the sample, wherein the combination of determined signals indicate the efficacy of an allergen-specific desensitization treatment of a subject with said allergen.

Based on the surprising finding that the expression of a basophil inhibitory marker on the surface of basophils is downregulated upon activation after being incubated with the allergen to which the subject is allergic, the efficacy of an allergen-specific desensitization treatment of a subject with said allergen can be indicated by the signal intensities of the labels of the combination of the basophil inhibitory and activation markers.

In one embodiment, the marker levels are determined by flow or mass cytometric measurement.

In embodiments, the basophil inhibitory marker is CD32.

In embodiments, the basophil activation marker is CD63.

In embodiments, all cell populations are considered in the context of the present invention.

Therefore, CD63 and CD32, as well as other markers, are detected not only in basophils but in all bins of the entire blood cell populations, e.g. also in eosinophils, neutrophils and monocytes. Therefore, different types of allergies are also identified, which predominantly show activation of neutrophils or eosinophils. In addition, specific bin patterns and other bin-based features collectively allow prognostic classification of responders and non-responders to immunotherapy.

In one embodiment, an affinity agent binding to receptor FcεRI on cell surfaces can be applied to IgE-type antibodies to mediate an allergy. Therefore, cell subpopulations with high levels of FcεRI are a significant indicator for diagnosing IgE-type mediated allergies.

CD63 is a commonly used basophil activation marker in BAT, which is usually used together with a basophil identification marker e.g., CCR3. However, the BAT assay comprising the use of a basophil activation marker and/or basophil identification marker can at times not distinguish between a stable and transient responder.

CD32 is also known as FcγRII. The FcγRII family consists of a family of primarily cell membrane receptor proteins. They are encoded by the mRNA splice variants of three highly related genes, CD32A, CD32B and CD32C, which arose by recombination of CD32A and CD32B. All members of the FcγRII family are integral membrane glycoproteins and contain conserved extracellular domains, exhibiting an overall 85% amino acid sequence identity. CD32A is found on platelets, neutrophils, macrophages, and dendritic cells (DCs), Langerhans cells, mast cells, basophils, eosinophils, monocytes, megakaryocytes, and a subpopulation of activated CD4+ T cells. CD32B is found on basophils, neutrophils, monocytes, and macrophages.

Surprisingly, the expression of CD32, as an exemplary basophil surface marker, is downregulated upon activation of basophils after being incubated with the allergen to which the subject is allergic. Based on this surprising finding, it is advantageous to employ a combination of a basophil activation marker, for example CD63, and a basophil inhibitory marker, for example CD32, in a BAT to determine the efficacy of allergen-specific desensitization treatment. Such an assay characterizes the function of basophils in reaction to an allergen in both activatory and inhibitory aspects.

In embodiments, the basophil inhibitory marker is CD32A. In embodiments, the basophil inhibitory marker is CD32B. In embodiments, the basophil inhibitory marker is CD32C. In one embodiment, the basophil inhibitory marker can be any combination of CD32A, CD32B and/or CD32C.

In one embodiment, the levels of the basophil inhibitory marker and basophil activation marker are negatively correlated. In embodiments, when the levels of the basophil inhibitory marker are greater than the basophil activation marker, said levels indicate an efficacy of an allergen-specific desensitization treatment.

In the context of the invention, in embodiments, the level of a marker refers to the absolute value of logarithmic fold change (|Ig^{fold change}|) of the signal intensities of the basophils. In one embodiment, if the absolute value of logarithmic fold change of the signal intensity of the basophil inhibitory marker is greater than the logarithmic fold change of the basophil activation marker, this indicates an efficacy of an allergen-specific desensitization treatment.

In one embodiment, the levels of the basophil markers can be presented as a colour code, for example, more yellow/red areas representing strong signal intensities/higher levels, and more green/blue areas representing weaker signal intensities/lower levels.

In one embodiment, the method as used herein comprises determining additionally a level of at least one basophil identification marker.

Exemplary basophil identification markers are selected from the group consisting of FcεRI, CCR3, IgE and/or CD123.

In one embodiment, the method as used herein comprises determining additionally a level of at least one basophil exclusion marker.

Exemplary basophil exclusion markers are CD66b, CD16, Siglec-8, CD14 and HLA-DR, preferably HLA-DR.

In one embodiment, the method as used herein comprises determining additionally a level of at least one eosinophil identification marker.

Exemplary eosinophil identification marker are CD23, CD14, CD66b, SSCA-high and Siglec-8, preferably Siglec-8.

In one embodiment, the method as used herein, comprises determining additionally a level of at least one neutrophil identification marker.

Exemplary neutrophil identification markers are CD66b, CD11 b and CD16, preferably CD16.

In embodiments, the method as used herein comprises carrying out the method at multiple time points. Such time points may occur either before, during or after therapy.

In embodiments, carrying out the method at multiple time points comprises conducting the method at a first time point on a sample obtained from the subject prior to or upon therapy initiation, and at a second time point on a sample obtained from the subject after having received an allergen-specific desensitization treatment.

In embodiments, the second time point is 1 to 6 weeks, preferably 2 to 4 weeks, after initiation of the allergen-specific desensitization treatment.

In embodiments, the multiple time points may additionally include any time point, beyond the first and second time points, between the initiation of the allergen-specific desensitization treatment and termination of the treatment, as well as or alternatively after termination of the treatment.

Conducing the assay as described herein enables a more accurate representation of the potential efficacy of the treatment. In particular, early analysis via the inventive test may indicate that the treatment is likely to be efficacious, and further treatment should continue. Improvements in predicted efficacy over time, using the inventive test, will also indicate continuance of the test over the coming weeks, months and/or years. On the other hand, indications of a lack of efficacy of the test, either initially or over repeated assessment after initiation of the therapy, indicate potential discontinuation of the therapy, saving time and money, and avoiding potential discomfort to the patient.

In embodiments, the method comprises determining a level of:
at least one basophil inhibitory marker, preferably CD32,
at least one basophil activation marker, preferably CD63,
at least one basophil identification marker, preferably FcεRI, and
at least one of a basophil exclusion marker, preferably HLA-DR, an eosinophil identification marker, preferably Siglec-8, and/or a neutrophil identification marker, preferably CD16, in the sample.

Through this combination of markers an improved method is obtained that is relatively straightforward and enables a more complete picture of the underlying immune response in the subject to be obtained.

In further embodiment, the method comprises determining a level of at least CD32, CD63 and FcεRI, and optionally HLA-DR in the sample.

The combination of markers at the single cell level allows specific subpopulations of cells to be grouped and visualized in appropriate purity. Some examples are:
CD32 and CD63: Combinatorial measurement of CD63 and CD32 at the single cell level allows identification of cell populations that differ into CD63 positive only or CD32 positive only (SP=single positive cells) and CD63 positive CD32 positive (DP=double positive cells). Increased frequencies of CD32-SP cells and lower frequencies of CD63-SP cells is one feature indicating response to immunotherapy.
FcεRI^{high} and HLA-DR^{neg}: FcεRI marker allows a good separation of basophiles from other cells. It can be improved in combination with the HLA-DR marker, because few antigen-presenting cells, such as monocytes and DCs (HLA-DR^{pos} cells, up to 5%) can be excluded in addition.
FcεRI^{high} and Siglec-8^{pos} or CD23^{pos} or CD123^{high} or SSCA^{high}: identification and characterisation of a specific population of eosinophils with very high affinity to IgE and/or IgG complexes.
CD63^{pos} and other cell specific markers such as Siglec-8 for eosinophils: CD63 can also be used as an activation marker for cell subpopulations other than basophils, such as eosinophils.
CD32^{pos} and other cell specific markers such as Siglec-8 for eosinophils: CD32 can also be used as inhibitory marker for cell subpopulations other than basophils, such as eosinophils.

In one embodiment, the method as used herein comprises:
a) incubating a blood sample from said subject with an allergen, against which the subject exhibits or is suspected to exhibit a hypersensitivity reaction, wherein the sample comprises basophils;
b) treating the sample with at least one label that specifically binds a basophil inhibitory marker, preferably CD32, and at least one label that specifically binds a basophil activation marker, preferably CD63;
c) determining the intensities of the labels in the sample;
d) grouping of the basophils according to a model trained on multiple feature sets, wherein each feature set comprises a combination of binned marker intensities of a basophil inhibitory marker and binned marker intensities of a basophil activation marker; and preferably binned marker intensities of a basophile identification marker, preferably binned marker intensities of the side scatter.
e) classifying the basophils according to step c) and determining and/or prognosing the efficacy of an allergen-specific desensitization treatment.

In embodiments, the model trained on multiple feature sets is a model developed using machine learning, in order to identify and improve the grouping of basophils based on the expression of basophil inhibitory marker(s) and basophil activation marker(s). In a preferred embodiment, the grouping of basophil can be further based on a basophil identification marker. In another preferred embodiment, the grouping of basophil can be further based on SSCA, i.e. side scatter.

In embodiments, each feature set comprises a combination of binned marker intensities of a basophil inhibitory marker and binned marker intensities of a basophil activation marker. These (at least) two sets of binned marker intensities are employed to generate a feature set, which is subsequently used to train the model employed in basophil grouping. By enhancing the model using these feature sets, the basophil inhibitory marker and basophil activation markers can be effectively interrogated in an automated manner.

It is advantageous that the machine learning-supported evaluation of the efficacy of allergen-specific desensitization treatment establishes an automatic workflow for an accurate evaluation of immunotherapy efficacy that may be applied in clinical and/or diagnostic practice. The workflow renders the flow cytometry analysis of BAT more transparent, reproducible and efficient.

Additionally, the automatic workflow enables the analysis of more than two fluorescent marker without repeated sub-gating.

The method as described in WO2020/201443 can be employed to generate the feature sets and train the model for gating/grouping, using machine learning. WO2020/201443 is hereby incorporated by reference. A more detailed representation of training the model is provided in the detailed description, below.

In embodiments, the machine learning model is trained with multiple pairs of feature sets. As explained in more detail below, the model is trained by machine learning for evaluating different feature sets, for example, a combination of binned signal intensities of a basophil inhibitory marker and a basophil activation marker, preferably CD63 and CD32, or a combination of two or more basophil activation marker and a basophil inhibitory marker, or a combination of a basophil activation marker, basophil inhibitory marker and a basophil identification marker.

The feature sets are not limited to these combinations and can be any combination of a basophil activation marker, a basophil inhibitory marker, a basophil identification marker, a basophil exclusion marker, an eosinophil identification marker and/or a neutrophil identification marker, wherein a basophil activation marker and a basophil inhibitory marker are required.

Another aspect of the invention relates to a system for determining and/or prognosing the efficacy of an allergen-specific desensitization treatment of a subject in a method as described herein, comprising: a data store comprising multiple feature sets; a software or an algorithm configured for generating the feature sets, comparing the feature sets, and visualising the gating/grouping results; and wherein the software or algorithm is configured to apply the machine learning model.

A further aspect of the invention relates to the use of an affinity reagent that specifically binds CD32 in an in vitro method for determining and/or prognosing an efficacy of an allergen-specific desensitization treatment of a subject.

In embodiments, all cell populations including but not limited to basophils, neutrophils, eosinophils and monocytes are considered in the context of the present invention. Detecting CD32 with an affinity regent can be applied not only to basophils but to all bins of the entire blood cell populations. Using CD32 as an inhibitory marker can be applied to all cell populations for determining different types of allergies. Other allergy types can be identified by analyzing further cell populations other than basophils, which predominantly show activation of neutrophils or eosinophils. In embodiments, specific bin patterns and other bin-based features collectively allow prognostic classification of responders and non-responders to immunotherapy.

In embodiments, the affinity reagent that specifically binds CD32 is used as a label to characterize the cells in a sample obtained from the subject.

In embodiments, the affinity reagent that specifically binds CD32 is used in combination with an affinity reagent that specifically binds CD63.

To the knowledge of the inventors, CD32, and thus an affinity reagent that specifically binds CD32, has not previously been employed in a method of determining and/or prognosing an efficacy of an allergen-specific desensitization treatment of a subject.

An affinity reagent that specifically binds to a surface marker, such as CD32, or CD63, is selected from but not limited to the group of an antibody, peptide, nucleic acid, or other small molecule that specifically binds that surface marker or any fragment(s) thereof in order to identify, track or capture its target molecule.

A further aspect of the invention relates to a kit for determining and/or prognosing the efficacy of an allergen-specific desensitization treatment of a subject in a method as used herein, comprising: an allergen against which the subject exhibits a hypersensitivity reaction; and at least one label that specifically binds a basophil inhibitory marker and at least one label that specifically binds a basophil activation marker.

In embodiments, the present invention relates to an in vitro method for determining basophil activation in a sample obtained from a subject, comprising treating said sample with an allergen and determining a level of at least one basophil inhibitory marker and at least one basophil activation marker in the sample, wherein the basophil inhibitory marker comprises CD32.

In embodiments, the basophil activation marker comprises CD63 is applied to the method as described herein.

In embodiments, the method as described herein comprises:
i) incubating a blood sample from said subject with an allergen, against which the subject exhibits or is suspected to exhibit a hypersensitivity reaction, wherein the sample comprises basophils;
ii) treating the sample with at least one label that specifically binds a basophil inhibitory marker and at least one label that specifically binds a basophil activation marker;
iii) determining the signal intensities and resulting frequencies of the labels in the sample, wherein the combination of determined signals indicates basophil activation in a sample.

In embodiments, the marker levels are determined by flow or mass cytometric measurement.

In embodiments, the levels of the basophil inhibitory marker and basophil activation marker are negatively correlated.

In embodiments, the method as described herein comprises determining additionally a level of at least one basophil identification marker selected from the group consisting of FcεRI, CCR3, IgE and/or CD123.

In embodiments, the method as described herein comprises determining additionally a level of at least one basophil exclusion marker, preferably HLA-DR.

In embodiments, the method as described herein comprises determining additionally a level of at least one eosinophil identification marker, preferably Siglec-8, and/or comprising determining additionally a level of at least one neutrophil identification marker, preferably CD66b.

In embodiments, the method as described herein comprises determining a level of
i) at least one basophil inhibitory marker comprising CD32,
ii) at least one basophil activation marker comprising CD63,
iii) at least one basophil identification marker, preferably FcεRI, and at least one of a basophil exclusion marker, preferably HLA-DR, an eosinophil identification marker, preferably Siglec-8, and/or a neutrophil identification marker, preferably CD66b, in the sample.

In embodiments, the method as described herein comprises determining a level of at least CD32, CD63 and FcεRI, and optionally HLA-DR in the sample.

In embodiments, method as described herein can be for determining and/or prognosing an efficacy of an allergen-specific desensitization treatment of a subject.

In embodiments, the method as described herein comprises determining and/or prognosing an efficacy of an allergen-specific desensitization treatment of a subject, the method comprising carrying out the method of the invention at multiple time points, comprising at a first time point on a sample obtained from the subject prior to or upon therapy initiation, and at a second time point on a sample obtained from the subject after having received an allergen-specific desensitization treatment, wherein the second time point is preferably 1 to 6 weeks, preferably 2 to 4 weeks, after initiation of the allergen-specific desensitization treatment.

In embodiments, the method as described herein comprises:
i) incubating a blood sample from said subject with an allergen, against which the subject exhibits or is suspected to exhibit a hypersensitivity reaction, wherein the sample comprises basophils;
ii) treating the sample with at least one label that specifically binds a basophil inhibitory marker CD32, and at least one label that specifically binds a basophil activation marker CD63;
iii) determining the intensities of the labels in the sample;
iv) grouping the basophils according to a model trained on multiple feature sets, wherein each feature set comprises a combination of binned marker intensities of a basophil inhibitory marker and binned marker intensities of a basophil activation marker preferably with a basophil identification marker; and
v) classifying the basophils according to step c) and determining and/or prognosing the efficacy of an allergen-specific desensitization treatment.

In embodiments, step b) above further comprises a label that specifically binds FcεRI.

In embodiments, the present invention relates to a system for determining basophil activation in a sample obtained from a subject in a method as described herein, comprising:
i) a data store comprising multiple feature sets;
ii) an algorithm configured for generating the feature sets, comparing the feature sets, and visualising the grouping results; and
iii) wherein the algorithm is configured to apply the model of claims 13-14.

In embodiments, the present invention relates to use of an affinity reagent that specifically binds CD32 in an in vitro method for determining basophil activation in a sample obtained from a subject.

In embodiments, the use of an affinity reagent that specifically binds CD32 in an in vitro method for determining basophil activation in a sample obtained from a subject, in combination with the use of an affinity reagent that specifically binds CD63.

In embodiments, the use of an affinity reagent that specifically binds CD32 in an in vitro method for determining basophil activation in a sample obtained from a subject, in combination with the use of an affinity reagent that specifically binds CD63 and with an affinity reagent that specifically binds FcεRI.

In embodiments, the present invention relates to a kit for determining basophil activation in a sample obtained from a subject in a method as used herein, comprising:
an allergen against which the subject exhibits a hypersensitivity reaction; and
at least one label that specifically binds a basophil inhibitory marker CD32, and at least one label that specifically binds a basophil activation marker, preferably CD63.

In embodiments, the kit as described herein comprises/consists of a label that specifically binds CD32 and a label that specifically binds CD63.

In embodiments, the kit as described herein comprises/consists of a label that specifically binds CD32, a label that specifically binds CD63 and a label that specifically binds FcεRI.

The embodiments describing the method for determining basophil activation (BAT) are equivalent embodiments to the method for determining and/or prognosing an efficacy of an allergen-specific desensitization treatment of a subject. Thus, all the advantage and technical effect of the method for determining and/or prognosing an efficacy of an allergen-specific desensitization treatment of a subject can be applied to the embodiments describing the method for determining basophil activation (BAT).

The embodiments describing the method of the invention may be used to describe the system of the invention, and vice versa. This also applies to any embodiments used to describe the method and the kit, or the system and the kit. The invention is unified by the novel and beneficial employment of a basophil activation marker and a basophil inhibitory marker in combination, and thus the relevant features described herein for one aspect may be used to describe any given aspect of the invention, in a manner in conformity with the understanding of a skilled person.

### DETAILED DESCRIPTION OF THE INVENTION

All cited documents of the patent and non-patent literature are hereby incorporated by reference in their entirety.

The invention relates to an in vitro method for determining and/or prognosing an efficacy of an allergen-specific desensitization treatment of a subject, comprising providing a sample from said subject, treating said sample with an allergen and determining a level of at least one basophil inhibitory marker and at least one basophil activation marker in the sample.

The term “allergen” refers to any agent, also referred to as an antigen, that produces an abnormally vigorous immune response. An allergen or antigen is capable of stimulating a hypersensitivity reaction in atopic individuals. The allergen in the context of the invention can be, without limitation, any test substance, preferably an antigen, a mitogen, a protein or peptide, a protein allergen or a peptide allergen, a group or mixture of protein and/or peptide allergens, a non-proteinaceous allergen, a low molecular weight allergen, a low molecular weight drug substance, or a hapten. In some embodiments the test substance is an antigen, hapten or allergen, wherein the antigen, hapten or allergen is a low molecular weight substance, preferably below 1000 Da. Allergy diagnostics include but are not limited to food allergy, insect venoms allergy, atopy, inhalation allergies which enter the body via the air the mucous membranes, such as, pollen from trees, grasses and herbs.

“Treating the sample with an allergen” shall mean contacting or incubating the sample with an allergen for any period of time, preferably for less than one hour.

The term “allergen-specific desensitization treatment” as used herein refers to a medical treatment for environmental allergies, such as insect bites, asthma and food, amongst others. It is an effective treatment used by allergists and immunologists for many common allergic conditions.

This form of therapy typically involves the subcutaneous administration of gradually increasing quantities of the patient's relevant allergens until a dose is reached that is effective in inducing immunological tolerance to the allergens. The primary objectives of allergen-specific immunotherapy are to decrease the symptoms triggered by allergens and to prevent recurrence of the disease in the long-term. Allergen-specific desensitization treatment is interchangeable with allergen-specific immunotherapy, allergen-specific hypersensitization and allergy shots.

In some embodiments, an efficacy of the allergen-specific therapy shall mean an induced immunologic tolerance to the allergen in the patient after a suitable dose of allergen is administered.

Human basophils are one class of leukocytes circulating in the blood stream and belong to the granulocytes. Despite their low abundance in human blood (less than 2% of the leukocyte fraction), they play a central role in allergic hypersensitivity reactions by releasing potent inflammatory mediators. Moreover, basophils represent the major interleukin-4 secreting human cell. Interleukin-4 μlays a key immunological role.

Immunoglobulin E (IgE) represents one of the classes of immunoglobulins. It is known to participate in allergic reactions. Circulating IgE molecules bind to the basophil membrane via the high affinity FcεRI receptor. An allergen, usually a protein of a molecular size of more than 5000 Da, is able to crosslink two neighboured IgE molecules bound on the basophils. By this crosslinking, a complex process is activated at the membrane level by an increase of membrane fluidity leading to IgE/FcεRI receptor clustering, degranulation of the cells and initiation of ionic fluxes into the basophilic cells which ultimately lead to the release of inflammatory mediators, such as histamine or sulfidoleukotrienes, as well as to the expression on, up-regulation at or migration of glycoproteins to the basophil membrane. Examples of such glycoproteins are CD45, CD63 or CD203c which are members of the so-called clustered differentiation (CD) antigens.

Non-IgE mediated reactions do also exist or are related to allergens for which an IgE mechanism has not been clearly established. The non-IgE mediated reactions are usually induced by low molecular weight substances such as a whole series of drugs, some food additives, other chemicals or agents, fMLP, complement factor C5a, etc. Both IgE and non-IgE mediated reactions may lead to basophil activation. Oppositely to IgE mediated basophil activation, the underlying pathophysiological mechanism for non-IgE mediated basophil activation and allergic reactions, respectively, is still not known to date.

The basophil activation test (BAT) is a functional assay that measures a degree of degranulation following stimulation with allergen or controls by flow cytometry. It correlates directly with histamine release. Basophil activation was first studied by quantification of the amount of mediator release (histamine and leukotriene C4) or by staining with specific fluorescent dyes and subsequent microscopic counting of fluorescent basophils. Later, the availability of flow cytometers capable of analyzing several thousand cells per second led to the development of several methods first based on basophil staining, for example with alcian blue. The BAT is thus typically a flow cytometry-based assay where the expression of activation markers is measured on the surface of basophils following stimulation with allergen. A positive basophil activation test can be seen as an in vitro surrogate of an acute allergic reaction in vivo. The BAT, being a functional assay, has the potential to resemble more closely the clinical phenotype of patients than allergy tests that merely detect the presence of allergen-specific IgE. In simple terms, the BAT can be seen as an OFC in a test tube, where instead of giving the food to a child by mouth, basophils involved in acute allergic reactions are exposed to a food extract in a test tube.

The term “basophil activation marker” also named “basophil activatory marker” refers to the surface marker which emerges or is upregulated/brought to the cell surface/presented on the cell surface/expressed in a cell and is contactable on the cell surface, after incubation of basophil with allergens or other triggers. The emergency or upregulation of the marker is measurable by flow or mass cytometry.

In one embodiment, a basophil activation marker is CD63. The expression of CD63 on basophil surface is increased upon activation of basophil by incubation with allergens or triggers. In one embodiment, a basophil activation marker is CD203c. In embodiments, the basophil activation marker is combination of CD63 and CD203c.

The term “basophil inhibitory marker” refers to a surface marker that inhibits or blocks basophil activation by interfering with activation signals or processes. Such a marker typically disappears, or is downregulated, or is expressed at lower levels, or is presented at lower levels on the cell surface, after incubation of basophil with allergens or other triggers. The level and down regulation of the marker is measurable by flow or mass cytometry. In the context of the present invention, an inhibitory marker is a functional inhibitor of basophil activation. An inhibitory marker can phenotypically disappear or be present in the same or in reduced amounts, for example be downregulated on the cell surface, as long as it is an inhibitor of basophil activation. For example, although the levels of CD123 are decreased upon basophil stimulation, it is not considered an inhibitor according to the present invention.

The term “basophil identification marker” refers to a surface marker for identifying basophils, including but not limited to FcεRI, CCR3, IgE, CD123 and CD203c. In one embodiment, for identifying basophils, any combination of the basophil identification markers can be used. In some embodiments, a combination of CD123 and CCR3 is used for identifying basophils.

The term “basophil exclusion marker” refers to a surface marker which is not, or is lowly expressed on the surface of a basophil, including but not limited to HLA-DR. In one embodiment, a combination of basophil identification marker and basophil exclusion marker is used for identifying basophils. In one embodiment, a combination of basophil identification marker and a cell property marker, side scatter values (SSCA), is used for identifying basophils.

CD32 is also known as FcγRII. The FcγRII family consists of a family of primarily cell membrane receptor proteins. They are encoded by the mRNA splice variants of three highly related genes —CD32A, CD32B and CD32C, which arose by recombination of CD32A and CD32B. All members of the FcγRII family are integral membrane glycoproteins and contain conserved extracellular domains, exhibiting an overall 85% amino acid identity. CD32A is found on platelets, neutrophils, macrophages, and dendritic cells (DCs), Langerhans cells, mast cells, basophils, eosinophils, monocytes, megakaryocytes, and a subpopulation of activated CD4+ T cells. CD32B is found on basophils, neutrophils, monocytes, and macrophages.

In one embodiment, a basophil inhibitory marker is CD32, whose expression levels on the basophil surface are typically reduced upon activation of basophil by incubation with allergens or triggers. In some embodiments, a basophil inhibitory marker is CD32A. In other embodiments, a basophil inhibitory marker is CD32B. In embodiments, the basophil inhibitory marker is CD32C. Any combinations are envisaged. Labels that bind one or more of these CD32 forms may be employed.

The term “flow cytometry” or “flow cytometric measurement” shall mean a technique used to detect and measure physical and/or chemical characteristics of a population of cells or particles. Flow cytometric measurement is used for e.g. cell counting, cell sorting, determining cell characteristics and function, detecting microorganisms, detecting biomarker, diagnosing disorders such as blood cancer. In this process, a sample containing cells or particles is suspended in a fluid and injected into a flow cytometer instrument. The sample is focused to ideally flow one cell at a time through a laser beam, where the light scattered is characteristic to the cells and their components. Cells are often labeled with fluorescent markers so light is absorbed and then emitted in a band of wavelengths. Tens of thousands of cells can be quickly examined, and the data gathered are processed by a computer.

Modern flow cytometers are able to analyze many thousands of particles per second, in “real time” and, if configured as cell sorters, can actively separate and isolate particles with specified optical properties at similar rates. A flow cytometer has five main components: a flow cell, a measuring system, a detector, an amplification system, and a computer for analysis of the signals. The flow cell has a liquid stream (sheath fluid), which carries and aligns the cells so that they pass single file through the light beam for sensing. The measuring system commonly uses measurement of impedance (or conductivity) and optical systems—lamps (mercury, xenon); high-power water-cooled lasers (argon, krypton, dye laser); low-power air-cooled lasers (argon (488 nm), red-HeNe (633 nm), green-HeNe, HeCd (UV)); diode lasers (blue, green, red, violet) resulting in light signals. The detector and analog-to-digital conversion (ADC) system converts analog measurements of forward-scattered light (FSC) and side-scattered light (SSC) as well as dye-specific fluorescence signals into digital signals that can be processed by a computer.

The term “mass cytometry” or “mass cytometric measurement” is a mass spectrometry technique based on inductively coupled plasma mass spectrometry and time of flight mass spectrometry used for the determination of the properties of cells (cytometry). In this approach, antibodies are conjugated with isotopically pure elements, and these antibodies are used to label cellular proteins. Cells are nebulized and sent through an argon plasma, which ionizes the metal-conjugated antibodies. The metal signals are then analyzed by a time-of-flight mass spectrometer. The approach overcomes limitations of spectral overlap in flow cytometry by utilizing discrete isotopes as a reporter system instead of traditional fluorophores which have broad emission spectra.

In embodiments, either flow cytometry or mass cytometry may be employed in the method, using appropriate labels and/or hardware.

The term “label” in the context of the invention refers to a detection reagent specifically binding to a target, such as a basophil activation marker or basophil inhibitory marker. The label can then be detected using e.g. flow cytometry or mass cytometry, and thus e.g. enable the determination of levels of basophil activation and/or inhibitory markers. In one embodiment, the label is a detection reagent which comprises a fluorescent dye and binds specifically to basophil activation or inhibitory marker. In embodiments, the label can be two or more detection reagents binding sequentially to the basophil activation marker wherein the detection reagents comprise at least a first detection reagent specifically binding to basophil activation or inhibitory marker and at least a second detection reagent with a fluorescent dye attaching to the first detection reagent.

The “detection reagent” or “label” as used herein are preferably reagents that are suitable to determine the herein described basophil marker(s), e.g. of CD32, CD63, FcεRI, CCR3, IgE and/or CD123. Such exemplary detection reagents are, for example, anti-CD32 antibodies, anti-CD63 antibodies, anti- FcεRI antibodies, anti-CCR3 antibodies, or anti-IgE antibodies or anti-CD123 antibodies. The label reagent refers also to a further detection reagent labeling the first detection reagent wherein the further detection reagent comprises a fluorescent dye including but not limited to Alexa Fluor® dyes, DyLight® Fluor dyes, Qdot® labels, R-phycoerythrin (R-PE), APC tandem dyes and biotin. Such labeling reagent might be used in flow cytometric analysis in the context of the invention. Further reagents that are employed in the flow cytometric analysis to determine the level of the marker(s) may also be comprised in the kit and are herein considered as detection reagents.

An affinity reagent that specifically binds to CD32 shall mean antibody, peptide, nucleic acid, or other small molecule with or without fluorescent dye that specifically binds CD32 or any fragment(s) thereof in order to identify, track, capture or influence its activity.

In the context of the invention, signal intensities of the labels are determinable by flow cytometry or other technique used to detect and measure physical and chemical characteristics of a population of cells or particles in the art, such as cellometer vision (Cytometry A. 2011 Jul; 79(7):507-17. doi:10.1002/cyto.a.21071. Epub 2011 Apr 29).

In one embodiment, the method as used herein, wherein the levels of the basophil inhibitory marker and basophil activation marker are negatively correlated within the selected grouping. The term “negatively correlated” shall mean that if an allergen-specific desensitization treatment is not effective, the basophil population isolated from the subject shows a high level of basophil activation marker and a low level of basophil inhibitory marker. If an allergen-specific desensitization treatment is effective, the basophil population isolated from the subject preferably shows a low level of basophil activation marker and a high level and frequency of basophil inhibitory marker.

A “hypersensitivity reaction” refers to undesirable reactions produced by the normal immune system, including allergies and autoimmunity. It further refers to as an overreaction of the immune system and these reactions may be damaging and uncomfortable. Hypersensitivity reactions can be classified into four types: type I—IgE mediated immediate reaction, type II- antibody-mediated cytotoxic reaction via IgG or IgM antibodies, type III- immune complex-mediated reaction, type IV—cell-mediated, delayed hypersensitivity reaction.

In one embodiment, the blood sample for use in the method as described herein is provided from the subject exhibits one or more types of hypersensitivity reactions against an allergen. In one embodiment, the subject exhibits type-I hypersensitivity reaction through IgE responses. In one embodiment, the subject exhibits a type IV-like hypersensitivity reaction in which the allergen directly (independently of IgE) promotes basophil and/or eosinophil reactivity.

The term “subject” or “patient” as used herein may be a vertebrate, preferably a mammal, more preferably a human subject. In embodiments, the term “subject” refers to a subject that is suspected of or diagnosed with an allergy, wherein e.g., said subject is not yet under allergy-specific desensitization treatment or in the middle of the treatment or after termination of the treatment.

The term “sample” shall mean a blood, serum, plasma sample. In embodiments, the sample comprises basophils.

The term “gating” refers to a basic conventional principle of flow cytometric analysis, which is the sequential identification and refinement of a cellular population of interest using one or more surface markers that are visualized, e.g., by fluorescence in a unique emission spectrum. The process of gating in flow cytometry typically refers to selecting an area on the scatter plot generated during the flow experiment that determines which cells are used for further analysis and which cells are not. That means, that cells that are not of interest will be “gated out”. In one embodiment, at least a basophil activation marker and at least a basophil inhibitory marker are used for gating the sample, wherein the sample comprises the basophils. In some embodiments, one or more basophil identification markers are used for gating the basophil population. In other embodiments, at least one basophil exclusion marker and/or at least one eosinophil identification marker and/or at least one neutrophil identification marker are used for gating the basophil population.

The term “grouping” refers to the characteristic pooling of cells with similar properties in the bin plots without “out-gating” of other cells but rather “out-grouping”. Therefore, areas of interest with specific properties can be compared to all other cells. In one embodiment patients with an unexpected allergen-induced neutrophil-subpopulation are identified as CD63^{pos} SSCA^{interm} FcεRI^{high}. “Grouping” comprises the cell population of both “gated in” and “gated out” wherein the cell population of “gated in” is with respect to basophil population which indicates whether the immune therapy succeeds or not.

The terms “grouping” and “gating” in the context of the analysis within one cell population or subpopulation, such as a basophil population or neutrophil population, are interchangeable. Both gated, e.g. a basophil population, and out-gated cell populations, e.g. a neutrophil subpopulation, can be grouped for further analysis.

The term “binned marker intensities” or “binned label intensities” as used herein refers to a collection of signal intensities of a label (or marker) exhibited by a cell population. A binned basophil marker shall mean that the signal intensities of a basophil marker of a cell population are collected. In one embodiment, the detected intensities of each label can be binned.

The term “feature set” as used herein refers to a combination of different features. In other words, a feature set is a combination of signal intensities of two or more basophil labels. For example, a feature set of a basophil population can be an intensity of CD63 combined with an intensity of CD32, or an intensity of CD63 combined with an intensity of CD32 and an intensity of FcεRI.

In one embodiment the feature set comprises at least an intensity of a basophil activation marker of a cell and at least an intensity of a basophil inhibitory marker of a cell. In embodiments, the feature set comprises further at least an intensity of a marker of a cell selected from basophil exclusion marker, eosinophil identification marker and neutrophil identification marker.

The training of the model used for grouping may be carried out as follows. A method for classifying selected label signals from cytometric measurements is carried out, comprising a first measurement and a second measurement. The first measurement comprises a measurement of a first sample, the second measurement is of a second sample. Each particle is labelled, as described herein, and each label is associated with an entity of the respective particles.

In embodiments, the method, as described in WO2020/201443, comprises the steps of:
a) binning the detected intensities of each label of the first measurement into a first number of bins,
b) generating at least one associated label function, such as a ratio of the detected intensities, in particular a ratio of the binned intensities, of two labels or a number of particles per bin of a label,
c) generating from the first measurement a number of first feature sets, each first feature set relating a combination of two binned label intensities to at least one associated label function,
d) binning the detected intensities of each label of the second measurement into a second number of bins,
e) generating at least one associated label function, such as a ratio of the detected intensities, in particular a ratio of the binned intensities, of two labels or a number of particles per bin of a label,
f) generating from the second measurement a number of second feature sets, each second feature set relating a combination of two binned label intensities to at least one associated label function,
wherein each first feature set and each second feature set comprising the same combination of label intensities or functions, form a pair of feature sets, and
g) providing the pairs of feature sets to a machine learning method, wherein the machine learning method determines at least one selected pair of feature sets that exhibits the largest variation between the first and the second feature set.

In embodiments, the measurements of the labels may comprise a detected intensity of a label associated to a specific entity for each particle. By measuring the intensity of the label associated to a certain entity, a concentration of the respective entity and/or a quantity of the particle can be determined. The detected intensity of a label may also be referred to as label intensity, label intensity or signal intensity in the context of the current specification.

In embodiments, the detected intensities of each label can be binned. This means that with respect to each label, a binned label intensity can be generated.

In embodiments, the detected intensities of a label can be binned in a serial manner, such that a 2-dimensional bin pattern is generated, i.e. the intensities of a first label can be binned, and in a subsequent step, the intensities of a second label can be binned. The combination of two binned label intensities defines a two-dimensional bin. In embodiments, a 2-dimensional binning is performed in one step providing a plurality of two-dimensional bins. With the term “2-dimensional binning” a binning of a combination of two label intensities is meant. In an embodiment, the one-step 2-dimensional binning is performed by arranging the intensities related to a first and a second label to each other and putting a grid comprising a plurality of equal sectors on the sorted intensities, wherein each sector of the grid represents one two-dimensional bin. Based on the intensities of the two binned label intensities, each particle can be related to a certain two-dimensional bin. By relating each particle to a respective bin, a reduction of the dimensionality can advantageously be provided.

In embodiments, at least one associated label function can be determined for each bin, in particular for each two-dimensional bin. A label function can be a ratio of intensities of two labels. In an embodiment, the label function is a ratio of binned intensities of two labels. The label function can be a number of particles per bin. According to an embodiment, the label function may be a statistical value with respect to a label intensity of a bin, in particular one of a mean value, a median, a minimum value, a maximum value, a standard deviation, a variance, an inter quartile range, a distance, a range, a correlation, or coefficient of variation.

In embodiments, a feature set relates a combination of two binned label intensities and at least one associated label function to each other. In other words, this means that a feature set can comprise three entries: a) a first binned label intensity related to a first entity, b) a second binned label intensity related to a second entity, and c) an associated label function (and/or a third label intensity related to the bin determined by the first and the second binned label intensity).

A feature set can be generated by a first measurement and is referred to as a first feature set. A second feature set can be a feature set generated by a second measurement. A first feature set and a second feature set that comprise the same combination of labels, intensities or functions form a pair of feature sets.

In an embodiment, for each combination of binned label intensities, a pair of feature sets is generated. A plurality of pairs of feature sets can be provided to a machine learning method to identify at least one selected pair of feature sets showing the largest variation between the first feature set and the second feature set. For example, all pairs of feature sets can be provided to a machine learning method. In other words, this means that based on all pairs of feature sets the machine learning method can determine the selected pair of feature sets showing the largest variation.

In an embodiment, a support vector machine approach is used to determine the largest variation between the first and the second feature set. Alternatively, a statistical value, such as a mean value, a variance, a range, a standard deviation and/or a cumulant can be determined for the first and the second feature set for each pair of feature sets, wherein the largest variation can be determined e.g. by a difference or ratio between the statistical value. An alternative embodiment is characterized in that an artificial neuronal network is used to determine the variation between the first and the second feature set. In an embodiment, the variation between the first and the second feature set is determined by means of a correlation.

By identifying the pair of feature sets showing the largest variation between the first feature set and the second feature set, the machine learning method can determine the label combination with which the first sample can be distinguished best from the second sample.

In an embodiment, the labels are identified that characterize a sample, in particular the cells or the particles of the sample, best. In other words, labels are identified that allow greatest discrimination of the labeled particles. An advantage of the method is that a most powerful label combination to discriminate between different samples is identified. The most powerful label combination is preferably the label combination whose associated pair of feature sets shows the largest variation between the first feature set and the second feature set.

The model used to gate the basophils is thus preferably trained by a method as described above, and as in WO2020/201443.

The term “automated gating/grouping” refers to gating/grouping a cell population of a sample according to selected markers without manual user input, for example by a model trained by machine learning.

The term “visualising the gating/grouping results” shall mean visualising the results of gating/grouping, for example visualizing the labelled cells on a biplot or a triplot or a plot in combination with colour code.

In one embodiment, the method as used herein comprising carrying out the method at multiple time points. By way of example, a first time point refers to a sample obtained from the subject prior to or upon therapy initiation, and at a second time point refers to a sample obtained from the subject after having received an allergen-specific desensitization treatment. In some embodiments, the second time point is preferably 1 to 6 weeks, preferably 2 to 4 weeks, after initiation of the allergen-specific desensitization treatment. Multiple time points shall mean any combination of time points prior to initiation of treatment, or between the initiation of the allergen-specific desensitization treatment and termination of the treatment, or after termination of the treatment, for example 2 months, 4 months, 8 months or 12 months after termination of the treatment.

The terms “determination” and “prognosis” in the context of the invention relates to the determination or prediction of an outcome, or specific effect, e.g., whether a subject is sensitized or still allergic to an allergen, against which the allergen specific desensitization treatment is directed. The terms “determination” and “prognosis” are not to be considered absolutely, i.e., a prognosed efficacy is not considered to predict with 100% certainty an efficacious outcome, rather the prognosis indicates a reasonable expectation of efficacy. Similarly, determination may also relate to determining, with a reasonable expectation of a skilled person, that an efficacy has occurred in the antigen-specific desensitization treatment.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of. Thus, the terms “comprising”/“including”/“having” mean that any further component (or likewise features, integers, steps and the like) can/may be present. The term “consisting of” means that no further component (or likewise features, integers, steps and the like) is present.

### FIGURES

The following figures are presented to describe particular embodiments of the invention, without being limiting in scope.

### BRIEF DESCRIPTION OF THE FIGURES

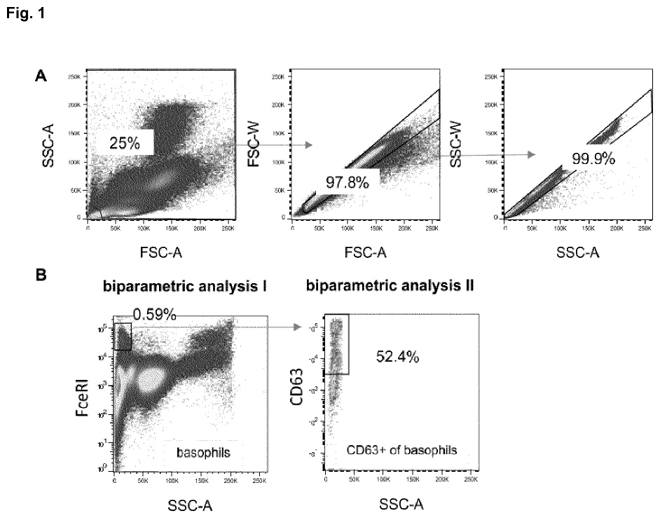

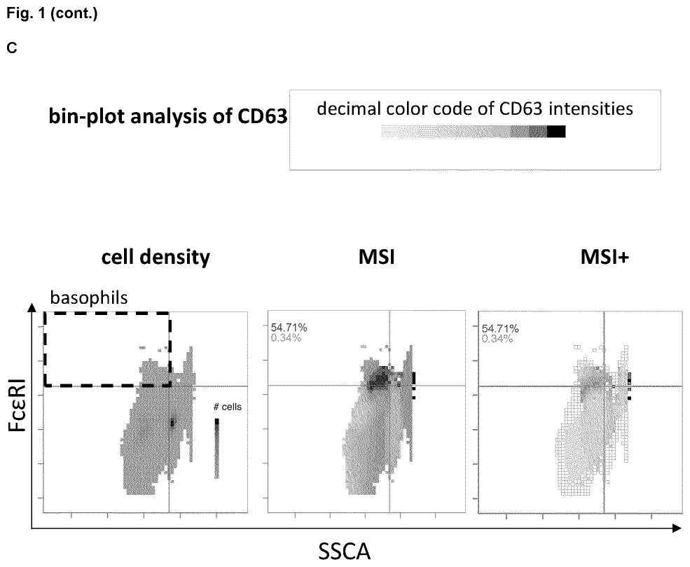

FIG. 1: Comparison of gating and grouping of whole blood cells for determination of activated basophiles.

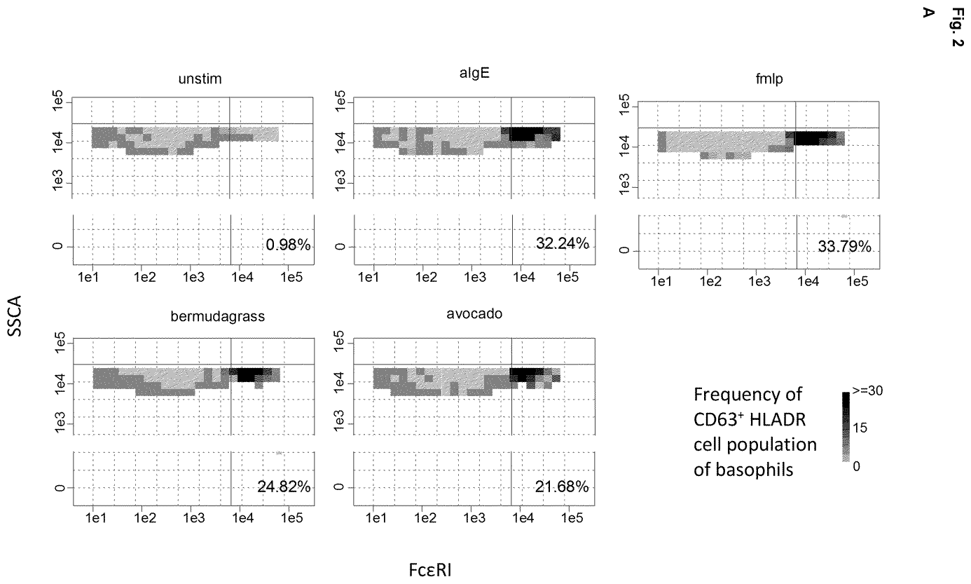

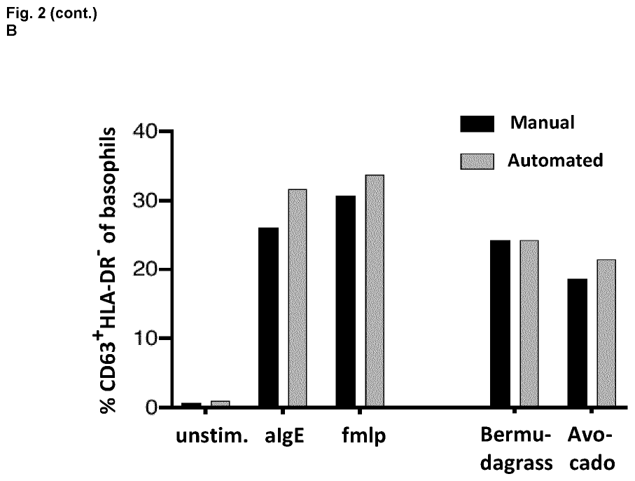

FIG. 2: Automatic four-parameter bin-plot analysis of BAT data.

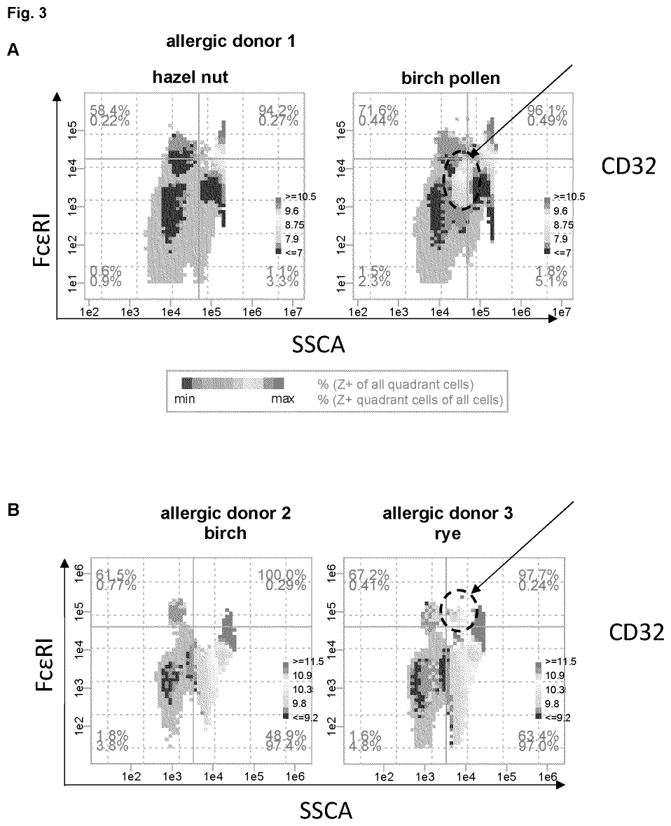

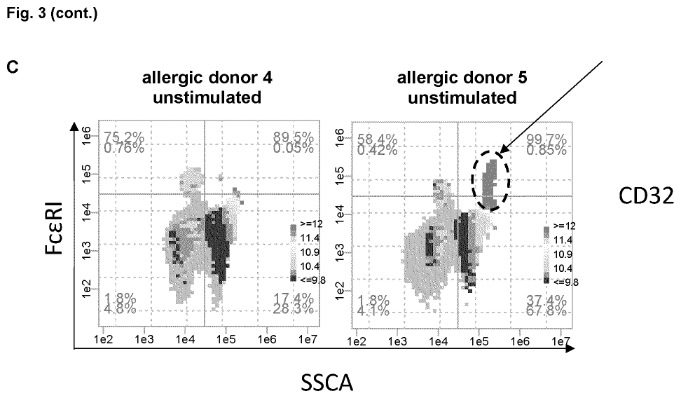

FIG. 3: Bin-based CD32 expression intensities reveal effects of immunotherapy and identify different allergy patterns.

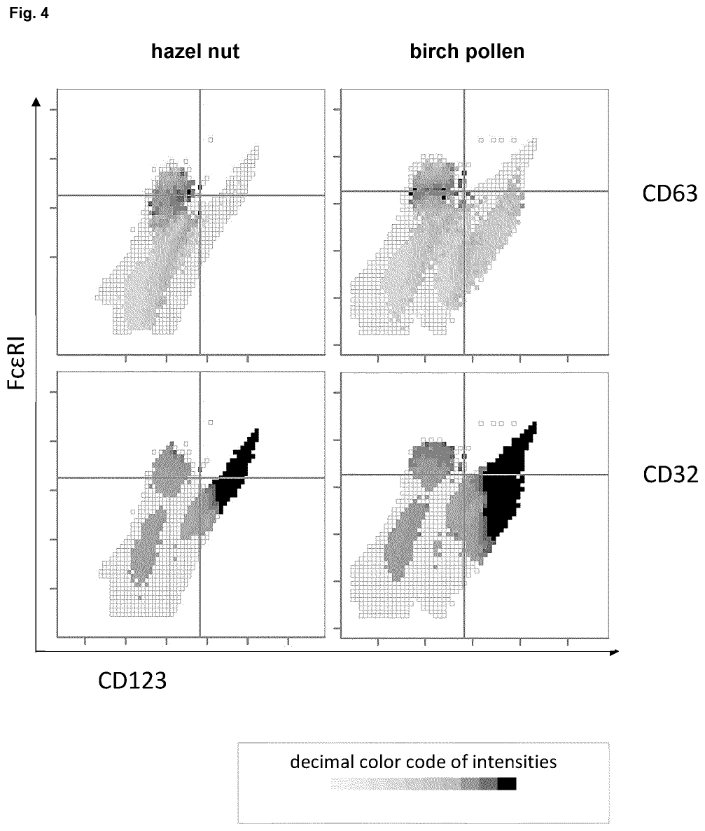

FIG. 4: Bin-based visualization of CD63 and CD32 expression intensities upon allergen stimulation with extracts of hazel nut and birch pollen, respectively.

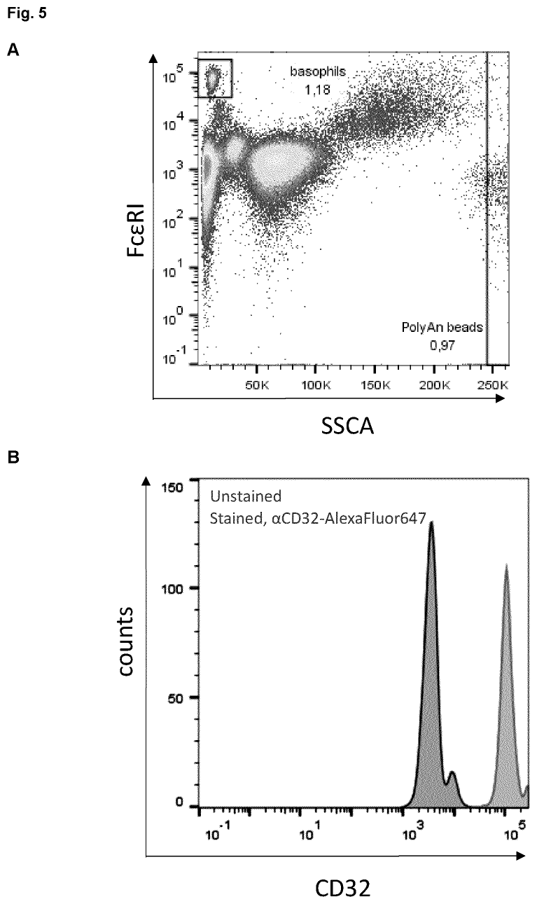

FIG. 5: CD32-protein coated beads for relative quantification of CD32 in BAT assays.

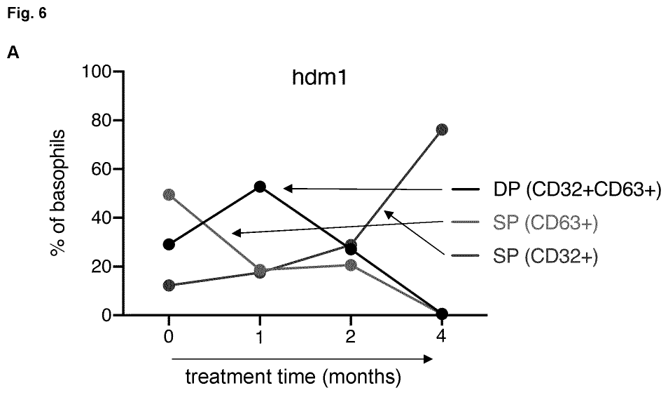

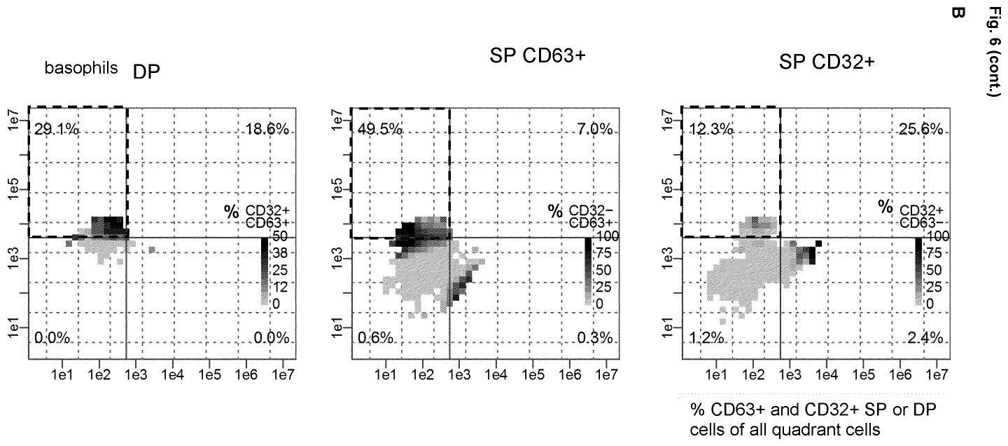

FIG. 6: Example of a successful immunotherapy.

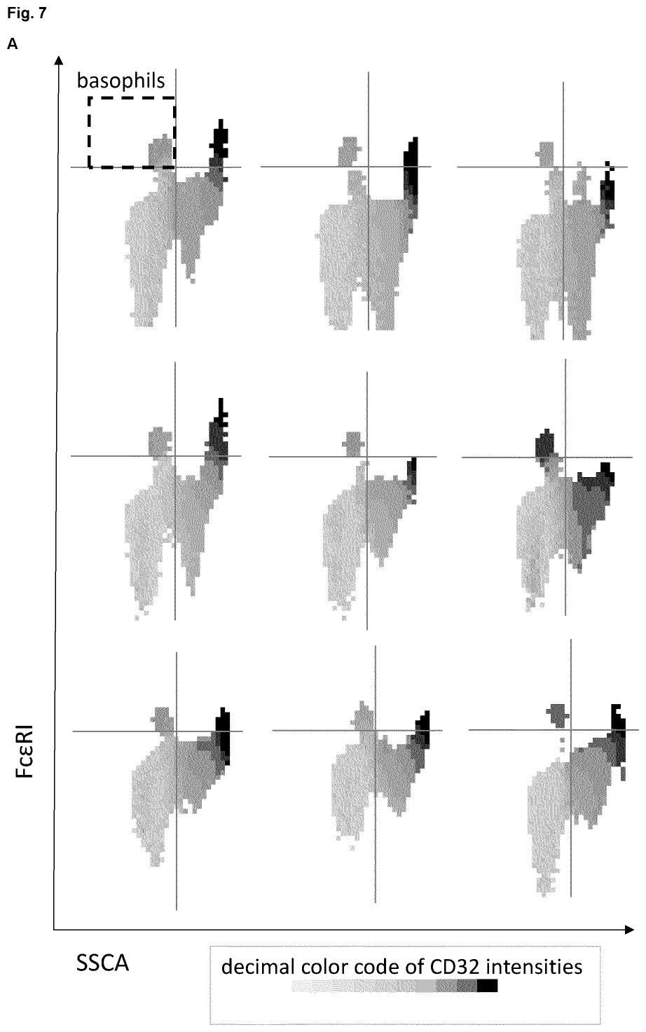

FIG. 7: CD32 bin intensities for identification of individual basophil quadrants.

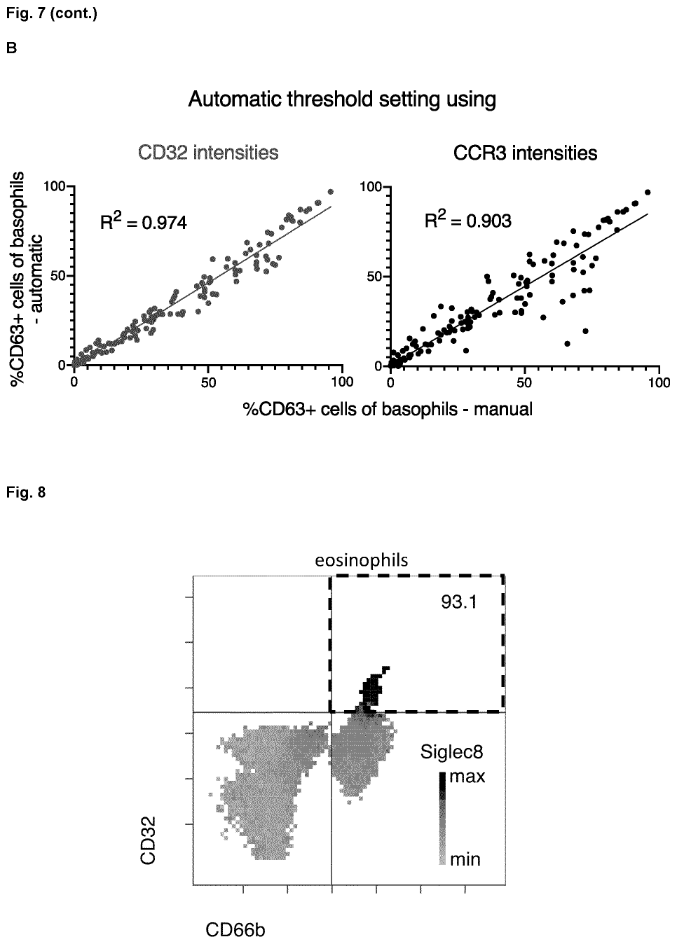

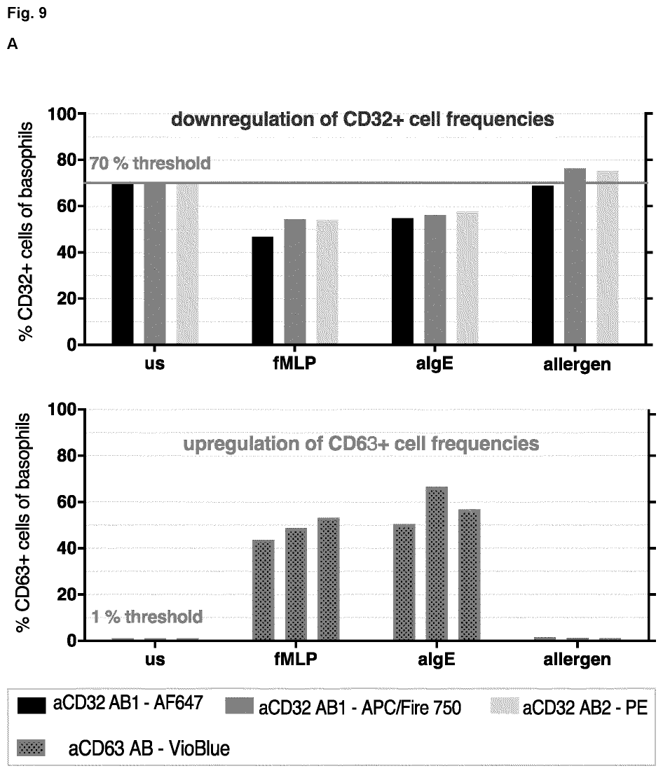

FIG. 8: CD32 bin intensities for identification of eosinophils.

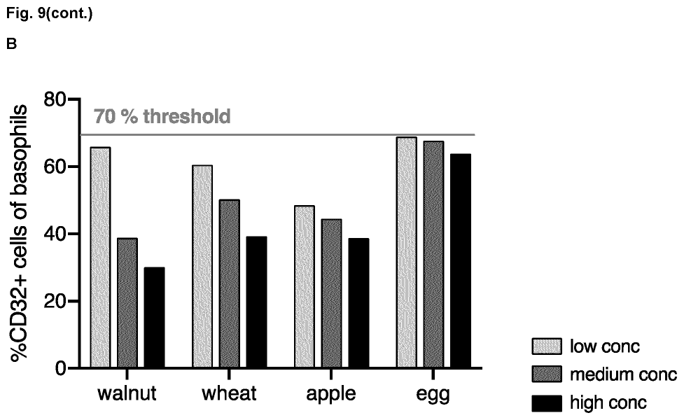

FIG. 9: Downregulation of CD32 intensities in positive controls and with individual allergens.

### DETAILED DESCRIPTION OF THE FIGURES

FIG. 1: Comparison of gating and grouping of whole blood cells for determination of activated basophiles.

(A) After a first common pre-gating procedure, the resulting live single cells were analyzed for the frequency of basophiles and CD63pos cells among the basophils with (B) a biparametric analysis and (C) bin-plot analysis.

FIG. 2: Automatic four-parameter bin-plot analysis of BAT data.

(A) Automatic bin-plot analysis of BAT data and visualization of the results. The frequencies of CD63⁺/HLA-DR⁻ cell population of basophils are shown. (B) The results of manual and automatic bin-plot analysis of a BAT experiment from one donor are presented.

FIG. 3: Bin-based CD32 expression intensities reveal effects of immunotherapy and identify different allergy patterns.

(A) CD32+ bin-pattern from samples of one allergic donor stimulated with two different allergens in one BAT experiment. The depicted area of CD32high cells (right plot) is kept from the unstimulated sample (not shown) after stimulation with the desensitizing allergen. (B) CD32+ bin-pattern from two allergic donors from the same BAT experiment. The depicted area of CD32^{interm}FcεRI^{high} cells is induced by allergens in one donor. (C) CD32+ bin-pattern from two allergic donors from the same BAT experiment. The depicted area of CD32^{high}FcεRI^{high} cells is present already in unstimulated samples of one donor and not changed by allergens.

FIG. 4: Bin-based visualization of CD63 and CD32 expression intensities upon allergen stimulation with extracts of hazel nut and birch pollen, respectively.

FIG. 5: CD32-protein coated beads for relative quantification of CD32 in BAT assays.

(A) Allergen-stimulated blood samples were stained (with labeled anti-CD32, anti-FcεRI, and anti-CD63 antibodies) and showed good separation from each other in the analysis. (B) Specific staining of beads coated with CD32 protein that were added to a non-stimulated and a stimulated blood sample is shown.

FIG. 6: Example of a successful immunotherapy.

(A) After stimulation of a blood sample with 0.1 μg hdm1 allergen, the frequencies of basophiles with different co-expression of the activation marker CD63 and the inhibition marker CD32 are shown in an allergic patient while desensitized to the house dust mite hdm1/2. (B) An example of 4 parametric bin plots to determine SP and DP basophiles at the onset of desensitization is presented. The frequencies are shown as color-coded bins in green intensity.

FIG. 7: CD32 bin intensities for identification of individual basophil quadrants.

(A) Example bin plots of 9 different donors from 7 different experiments show individual whole blood patterns using bin-plots with SSCA, FcεRI, and CD32.

(B) The results of automatic and manual bin-plot analysis of BAT data as well as CD32 and CCR3 as markers are compared (n=179).

FIG. 8: CD32 bin intensities for identification of eosinophils.

Exemplary bin-plot analysis shows that eosinophils characterized by Siglec8high/+ are CD32+.

FIG. 9: Downregulation of CD32 intensities in positive controls and with individual allergens.

(A) A nonallergic donor shows downregulation of CD32 expression intensity in positive controls (fmlp and algE) to a similar extent with 2 different CD32 antibodies and 3 different fluorescent markers shown as frequency of CD32+ cells.

(B) An allergic donor shows a concentration-dependent downregulation of CD32 intensities for detected food allergens (walnut 1 μg, 10 μg, 100 μg/ml; wheat extract 50 μg, 100 μg, 500 μg/ml; apple 1 μg, 10 μg, 100 μg/ml), but not for a non-allergenic food (egg 1 μg, 10 μg, 100 μg/ml) shown as frequency of CD32+ cells.

### Examples

### Example 1: Comparison of Gatinq and Groupning of Whole Blood Cells for Determination of Activated Basophiles

The basophil activation test (BAT) is in essence an allergy reaction in a test tube. In vitro allergen-stimulated whole blood samples were stained with labeled antibodies and measured by flow cytometry. For all kinds of analysis, there is a first pre-gating procedure (for both, manual and automatic processes) which is gating out cell fragments, dead cells, doublets or clumps (FIG. 1A). The selected living cells and their fluorescence intensity signals of markers are stored in a fcs-file (structured as a table with one cell per row and signal intensities per marker for this cell in the columns) for subsequent analysis either by conventional bi-parametric approach (FIG. 1B) or bin-based approach (FIG. 1C). With the bi-parametric approach, basophiles were selected by the FcεRI^{high} SSCA^{low} gate (FIG. 1B, left) and analyzed then for CD63^{pos} cells within the basophiles (FIG. 1B, right). With the bin-plotting, basophiles were grouped into the upper left quadrant (FcεRI^{high} SSCA^{low}) and CD63pos cells were analyzed not only in the basophile quadrant but also in the context of all other cells. All possible statistics of the grouped cells per bin can be analyzed. Depicted are cell density per bin, mean signal intensity (MSI) of a third marker (here CD63) per bin and mean signal intensity of a third markerjust among the third marker positive cells (MSI+, CD63+). Bins with less than 5 MSI+ cells are shown in grey.

There are similar results seen by both approaches (FIG. 1B vs FIG. 1C) for frequencies of basophiles among the blood cells (0.59% vs. 0.61% black number in the quadrant corner of the left plot) and CD63+ among the basophils (52.4% vs. 54.7% red number in the quadrant corner of the middle and right plot). In addition, bin plotting shows that there are also CD63+ cells (12.1%) in the right upper quadrant (eosinophils with high FcεRI) but these cells are lower in intensity than the CD63+ from the basophils (left upper quadrant).

### Example 2: Automatic Four-Parameter Bin-Plot Analysis of BAT Data

An automatic gating pipeline was used for pre-gating and this time also out-gating of SSCA^{high} cells in this example (FIG. 2A). Here, only CD63+ HLA-DR- cells were counted as activated basophiles in the right lower quadrant. The area of basophiles is demarked by the green area of the positive controls (algE and fmlp). The negative control (unstimulated sample) was set on having 1% CD63+ HLA-DR- cells in the basophil quadrant (right lower quadrant). Bins with less than 5 CD63+ HLA-DR cells are shown in grey.

There is a good agreement of manual and automatic results for frequencies of activated basophiles.

The automated gating plots of a BAT assay from an allergic patient reacting to Bermudagrass and avocado are depicted. An unstimulated blood sample is used as a negative control and an anti-IgE- or a fmlp-stimulated blood samples are used as positive control. The numbers in red indicate the frequencies of CD63+/HLA-DR-cells in the basophil quadrant. The results are shown in FIG. 2A.

The results generated from manual and automated gating of basophil activation, namely frequency of CD63+ basophils, in both controls and allergens, are compared. As shown in FIG. 2B they are consistent. The percentage of CD63 positive cell population from manual and automated gating are comparable.

### Example 3: Bin-Based CD32 Expression Intensities Reveal Effects of Immunotherapy and Identify Different Allergy Patterns

Manual BAT analysis using 3 parameters shows bin plotting of samples from the same experiment each (A-C). CD32+ intensities, visualized by bin-plotting, allows (together with CD63+ patterns) to identify different types of cell subsets which are induced by or unchanged after allergen-specific stimulation.

(A) An allergic donor with desensitization treatment for 6 months against birch pollen keeps the CD32signal height and frequency of the unstimulated sample within the basophile quadrant and the depicted area, respectively. Additional staining suggests a CD63^{neg} neutrophil subpopulation.

(B) Some allergic donors show different CD32+ bin-pattern than others in the BAT analysis. There appears an allergen-induced subpopulation with high intensities of CD63 and FcεRI.

(C) Many severe allergic donors show a CD32+ bin-pattern having a CD32^{high}FcεRI^{high}SSCA^{high} subpopulation in the BAT analysis. Additional staining suggests a CD63^{neg} eosinophil subpopulation.

### Summary of the Results

There were already three interesting sub-populations identified in allergic donors. First, a neutrophil subset (FIG. 3A) which is CD32^{high}FcεRI^{interm}SSCA^{low}CD63^{neg} where CD32 is not downregulated after birch-stimulation but after hazel-stimulation. Second, a neutrophil subset (FIG. 3B) is CD32^{interm}FcεRI^{high}SSCA^{interm}CD63high and induced by allergen-specific stimulation. Third, an eosinophil subpopulation (FIG. 3C) is CD32^{high}FcεRI^{high}SSCA^{high}CD63^{neg} and sometimes shows even higher numbers (green frequencies in the quadrants) than basophiles. This subpopulation is sometimes decreased by allergen-specific stimulation. It ranges in the donors of FIG. 3 (donor 1-5) between 0.05% and 0.85% of all blood cells.

### Example 4: Bin-Based Visualization of CD63 and CD32 Expression Intensities Upon Allergen Stimulation with Extracts of Hazel Nut and Birch Pollen, Respectively

With reference to FIG. 4, a population of basophils are observed which is SSCA^{low}/FcεRI^{high} and located in upper left quadrant. Upon stimulation, the expression intensities of CD63 are supposed to become stronger and CD32 become weaker.

Blood samples from an allergic patient who is immunized (desensitized) by birch pollen but not hazelnut pollen are under examination.

After stimulating the sample with birch pollen allergen, only a small number of basophil population exhibits a high intensity of CD63 (darker area, upper left) whereas a great number of basophil population exhibits a high intensity of CD32 (darker area, upper and lower right). The intensities of CD63 and CD32 are negatively correlated.

After stimulating the sample with hazelnut allergen, a large number of basophil population exhibits a high intensity of CD63 (darker area, upper left) whereas a small number of basophil population exhibits a high intensity of CD32 (darker area, upper and lower right). The intensities of CD63 and CD32 are negatively correlated.

The bin areas of the highest intensity (darker area) differ:
CD63^{high}=FcεRI^{low}, CD32^{high}=FcεRI^{high}

Main results:
neg. correlation of CD63 and CD32 bins
pos. correlation of FcεRI with CD32
more pos. CD32 bins in the basophile area (upper left quadrant) with birch pollen due to desensitization

TABLE 1
General overview of the markers:

| Receptor/Marker | Examples |
|---|---|
| Activation receptor | CD63, CD203c |
| Inhibitory receptor | CD32 |
| Basophil Marker | CD123 low/neg; CCR3 high, FcεRI high; SSCA |
| | low (Scatter-Marker), CD203C high; CRTH2 |
| | neg, HLA-DR neg |
| Eosinophile Marker | Siglec-8 pos; CD16 neg; CD66b pos; CD14 pos; |
| | SSCA high; CD23 pos |
| Neutrophile Marker | CD66b pos; SSCA pos; CD16 pos; CD11b pos |

### Example 5: CD32-Protein Coated Beads for Relative Quantification of CD32 in BAT Assays

For internal calibration of CD32 intensities, recombinant CD32 protein was biotinylated and coupled to streptavidin-coated beads (PMMA=poly methyl methacrylate beads, 16 μm), which are larger than the cells. The beads, along with the cells, are stained with labeled antibodies against CD32 and other markers after allergen stimulation of the blood samples.

(A) With reference to FIG. 5, the depicted plot shows a good separation of beads from all cells by size (SSCA) and no unspecific staining of the beads with anti-FcεRI antibodies (low intensity in this marker).

(B) Specific staining of beads coated with CD32 protein is represented by the shift in CD32 intensity due to anti-CD32 antibody staining compared to an unstained sample.

### Example 6: Example of a Successful Immunotherapy

Using 4-parametric plots, the frequencies of basophiles (upper left quadrant) with different co-expression of an activation marker (CD63) and an inhibition marker (CD32) were determined: single positive cells for CD63+(no CD32 expression) and for CD32 (no CD63 expression) as well as double positive cells which are positive for both markers. A time frame of 4 months of desensitization therapy of a donor with a house dust mite preparation against hdm1/2 is depicted.

After stimulation of a blood sample with 0.1 μg hdm1 allergen, the frequencies of basophiles with different co-expression of the activation marker CD63 and the inhibitory marker CD32 are shown in an allergic patient while desensitized to the house dust mite hdm1/2 (FIG. 6A).

An example of 4 parametric bin plots to determine SP and DP basophiles at the onset of desensitization is presented. The frequencies are shown as color-coded bins in green intensity.

An indication of successful therapy is shown here, because both processes can be observed, downregulation of SP and DP cells with the activation marker and upregulation of SP cells with an inhibitory marker (FIG. 6B).

### Example 7: Added Value of Using CD32 as a Marker Together with FcεRI and CD63

The basophil population and other cell populations or subpopulations, such as eosinophils, can be identified by using CD32 as a marker, preferably together with FcεRI and CD63. The assessment of the severity of allergy and the therapeutic success of immunotherapy may be based on the allergen-induced (or fmlp- or algE-induced) upregulation of CD63 and downregulation of CD32 expression intensities, which is often shown as a changed frequency of CD63+ and/or CD32+ cells. Data is presented in FIG. 7-9.

### Material and Methods for the Experimental Data Shown in FIG. 7-9:

### Assay and Markers

Lithium heparinized whole blood from healthy donors and allergic patients was used for the modified basophil activation test (BAT), using at least the three markers FcεRI, CD32 and CD63. Additional markers (notably HLADR, CD66b, CD11b, CD10) have been used to identify other populations and subpopulations, e.g. Siglec8 and CD66b for eosinophils. The markers CD123 and CCR3 were used to compare the results of our modified BAT with those of the conventional BAT.

### Materials

All allergens were from DST (Diagnostische Systeme & Technologien GmbH; Schwerin, Germany). Anti-human IgE (algE, Bethyl Laboratories) and N-Formyl-Met-Leu-Phe (fMLP, Tocris) were used as stimulants for the positive controls. The used marker antibodies were CD32-Alexa Fluor 647 (FUN-2, Biolegend), FcεRI-PE-Vio770 (CRA1, Miltenyi) and CD63-VioBlue (H5C6, Miltenyi). Further antibodies were CD123-PerCP-Vio700 (AC145, Miltenyi) or CCR3-PerCP-Cy5.5 (5EB, Miltenyi) for comparison of BAT results and HLADR-BV570 (L243), CD66b-FITC (G10F5), CD11 b-APC-Fire750 (CBRM1/5), CD10-BV605 (H110a) (all Biolegend) for identification of cell populations and subpopulations other than basophils.

### Assay Procedure

Each BAT sample (100 μl) is placed in a well of a 96 well v-bottom microtiter plate and consists of 50 μl whole blood plus RPMI medium, CD63 antibody solution, and allergen extract (usually final concentration 1 μg/ml unless otherwise specified) or PBS or stimulation premix (final concentration 2.5 μg/ml fmlp or 2.5 μM algE). After incubation at 37° C. for 15 min, the cells of the samples were fixed and lysed twice with Phosflow Lyse/Fix buffer (BD Biosciences). They were then washed and centrifuged twice with FACS buffer (1500 rpm for 3 minutes). The resuspended pellet was stained with FcεRI and CD32 antibody solution (or one or more other antibodies) for 20 min on ice, washed 2 times with 250 μl FACS buffer and resuspended in 70 μl FACS buffer for measurement with MACSQuant16 (Miltenyi) or LSRFortessa (BD Biosciences).

### Bin-Based Analysis and Bin Plot Visualization

To use our bin-based analysis workflow (described in detail in Hoang et al. Front Immunol. 2022, PMID: 35592315), the whole blood flow cytometry data from the modified BAT are imported as FCS files into the PRI analysis workflow. There, fluorescence intensities are transformed with the inverse hyperbolic sine (arcsinh) commonly used for flow cytometric analysis, with a cofactor of 1.

The signal intensities of two markers were divided into bins of width 0.2×0.2 asinh along the x and y axis for bin plot visualization. For all cells in each bin, different statistical values were calculated and displayed, usually in a color-coded manner as heat maps (grey scaling may also be employed, as shown here). For example, low values are represented as shades of blue, median values as yellow, and high values as red color.

The patterns of mean signal intensity of parameter z+ cells (MSI+) and the mean signal intensity of parameter z for all cells per bin (MSI) are analyzed. Additional statistics are percentage numbers in each quadrant. All events (cells) are included in the analysis but only bins containing five or more cells are displayed, balancing the impact of identifying comparatively rare subpopulations while retaining statistical robustness.

Usually bin-data were visualized as 3-parametric plots but sometimes as 4-parametric plots (e.g. FIG. 6B).

### SUMMARY

By way of example, the novel marker CD32, together with FcεRI and side scatter (SSCA), allows very effective grouping of basophils in a separate quadrant using unstimulated samples (us, negative controls) (FIG. 7). Furthermore, CD32 is downregulated only in positive control samples (FIG. 9A) or in allergen samples (FIG. 9B). After stimulation of a sample with a given concentration of an allergen, a higher percentage of CD32-positive basophils compared to CD63-positive or double-positive basophils (CD32+ and CD63+) indicates successful immunotherapy (FIG. 6).

Our new approach, combining modified BAT markers and bin-based whole blood data analysis, without gating but with grouping of basophils, allows sensitive, reproducible and automated determination and visualization of allergen sensitivities and successful monitoring of immunotherapies.

1. An in vitro method for determining basophil activation in a sample obtained from a subject, comprising treating said sample with an allergen and determining a level of at least one basophil inhibitory marker and at least one basophil activation marker in the sample, wherein the basophil inhibitory marker comprises CD32.

2. The method according to claim 1, wherein the basophil activation marker comprises CD63.

3. The method according to claim 1, comprising:
a) incubating a blood sample from said subject with an allergen, against which the subject exhibits or is suspected to exhibit a hypersensitivity reaction, wherein the sample comprises basophils;
b) treating the sample with at least one label that specifically binds a basophil inhibitory marker and at least one label that specifically binds a basophil activation marker;
c) determining the signal intensities of the labels in the sample, wherein the combination of determined signals indicates basophil activation in a sample.

4. The method according to claim 1, wherein the marker levels are determined by flow or mass cytometric measurement.

5. The method according to claim 1, wherein the levels of the basophil inhibitory marker and basophil activation marker are negatively correlated.

6. The method according to claim 1, comprising determining additionally a level of at least one basophil identification marker selected from the group consisting of FCERI, CCR3, IgE and CD123.

7. The method according to claim 1, comprising determining additionally a level of at least one basophil exclusion marker.

8. The method according to claim 1, comprising determining additionally a level of at least one eosinophil identification marker and/or comprising determining additionally a level of at least one neutrophil identification marker.

9. The method according to claim 1, comprising determining a level of
a) at least one basophil inhibitory marker comprising CD32,
b) at least one basophil activation marker comprising CD63,
c) at least one basophil identification marker, comprising FCERI, and
d) at least one of a basophil exclusion marker, HLA-DR, an eosinophil identification marker, Siglec-8, and/or a neutrophil identification marker, CD66b, in the sample.

10. The method according to claim 9, comprising determining a level of at least CD32, CD63 and FCERI in the sample.

11. The method according to claim 1, for determining an efficacy of an allergen-specific desensitization treatment of a subject.

12. The method according to claim 11, comprising carrying out the method of claim 1 at multiple time points with respect to an allergen-specific desensitization treatment, comprising at a first time point on a sample obtained from the subject prior to or upon therapy initiation, and at a second time point on a sample obtained from the subject after having received an allergen-specific desensitization treatment.

13. The method according to claim 1, comprising:
a) incubating a blood sample from said subject with an allergen, against which the subject exhibits or is suspected to exhibit a hypersensitivity reaction, wherein the sample comprises basophils;
b) treating the sample with at least one label that specifically binds a basophil inhibitory marker CD32, and at least one label that specifically binds a basophil activation marker CD63;
c) determining the intensities of the labels in the sample;
d) grouping the basophils according to a model trained on multiple feature sets, wherein each feature set comprises a combination of binned marker intensities of a basophil inhibitory marker and binned marker intensities of a basophil activation marker; and
e) classifying the basophils according to step c) and determining the efficacy of an allergen-specific desensitization treatment.

14. The method according to claim 13, wherein treating the sample further comprises a label that specifically binds FCERI.

15. A system for determining basophil activation in a sample obtained from subject in a method according to claim 1, comprising:
a) a data store comprising multiple feature sets;
b) an algorithm configured for generating the feature sets, comparing the feature sets, and visualising the grouping results; and
c) wherein the algorithm is configured to apply the model of claim 13.

16-18. (canceled)

19. A kit for determining basophil activation in a sample obtained from a subject in a method according to claim 1, comprising:
an allergen against which the subject exhibits a hypersensitivity reaction; and
at least one label that specifically binds a basophil inhibitory marker CD32, and at least one label that specifically binds a basophil activation marker.

20. The kit according to claim 19, further comprising a label that specifically binds CD32 and a label that specifically binds CD63.

21. The kit according to claim 19, further comprising a label that specifically binds CD32, a label that specifically binds CD63 and a label that specifically binds FCERI.
